(19) United States
(12) Patent Application Publication
(10) Pub. No.: US 2007/0038153 A1
(43) Pub. Date: Feb. 15, 2007

(54) Providing input associations for a computer system

(76) Inventors: Sara H. Basson, White Plains, NY (US); Alexander Faisman, Croton-on-Hudson, NY (US); Dimitri Kanevsky, Ossining, NY (US)

Correspondence Address: SCHMEISER, OLSEN & WATTS, 22 CENTURY HILL DRIVE, SUITE 302, LATHAM, NY, 12110, US

(21) Appl. No.: 11/194,332

(22) Filed: Aug. 1, 2005

(51) Int. Cl. A61B 5/103; G06K 9/00

(52) U.S. Cl. 600/595; 128/922; 382/128

(57) ABSTRACT

An association system and method. The distribution system comprises a computing system connected to a video input apparatus. The video input apparatus is adapted to send image data to the computing system. An exercise routine is developed for a user of the computing system. A first function to be performed by the computing system is associated with a first specified physical movement to be performed by the user of the computing system. The first specified physical movement is part of the exercise routine. The user performs the first specified physical movement. The first specified physical movement is detected by the video input apparatus. A first image of the first specified physical movement is transmitted by the video input apparatus to the computing system. The computing system performs the first function.

### BACKGROUND OF THE INVENTION

1. Technical Field

The present invention relates to a system and associated method for providing input associations in a computer system.

2. Related Art

Providing input to a processing system is typically limits a user to very few physical movements which may in turn negatively affect the user's health. Therefore there exists a need for devices to allow a user of a processing system to provide input for a processing system while allowing the user to engage in a plurality of physical movements.

### SUMMARY OF THE INVENTION

The present invention provides a method, comprising:

providing a computing system connected to a video input apparatus, said video input apparatus adapted to send image data to said computing system;

developing an exercise routine for a user of said computing system;

associating a first function to be performed by said computing system with a first specified physical movement to be performed by said user of said computing system, said first specified physical movement being part of said exercise routine;

performing by said user, said first specified physical movement;

detecting by said video input apparatus, said first specified physical movement; and

transmitting by said video input apparatus to computing system, a first image of said first specified physical movement; and

performing by said computing system, said first function.

The present invention provides a method, comprising:

providing a computing system connected to a first exercise apparatus, said first exercise apparatus adapted to send a first signal to said computing system upon using said first exercise apparatus;

developing an exercise routine for a user of said computing system;

associating a first function to be performed by said computing system with said first signal from said first exercise apparatus, said first exercise apparatus being associated with said exercise routine;

using by a user, said first exercise apparatus as part of said exercise routine;

receiving by said computing system, said first signal; and

performing by said computing system, said first function.

The present invention provides a computing system comprising a processor coupled to a computer-readable memory unit, said memory unit containing instructions that when executed by the processor implement a method for performing by said computing system, at least one function, said method comprising;

providing a video input apparatus connected to said computing system, said video input apparatus adapted to send image data to said computing system;

developing an exercise routine for a user of said computing system;
associating a first function to be performed by said computing system with a first specified physical movement to be performed by said user of said computing system, said first specified physical movement being part of said exercise routine;

performing by said user, said first specified physical movement;

detecting by said video input apparatus, said first specified physical movement; and

transmitting by said video input apparatus to said computing system, a first image of said first specified physical movement; and

performing by said computing system, said first function.

The present invention provides a computing system comprising a processor coupled to a computer-readable memory unit, said memory unit containing instructions that when executed by the processor implement a method for performing by said computing system, at least one function, said method comprising;

providing a first exercise apparatus connected to said computing system, said first exercise apparatus adapted to send a first signal to said computing system upon using said first exercise apparatus;

developing an exercise routine for a user of said computing system;

associating a first function to be performed by said computing system with said first signal from said first exercise apparatus, said first exercise apparatus being associated with said exercise routine;

using by a user, said first exercise apparatus as part of said exercise routine;

receiving by said computing system, said first signal; and

performing by said computing system, said first function.

The present invention advantageously provides a system and associated method for devices to allow a user of a processing system to provide input for a processing system while allowing the user to engage in a plurality of physical movements.

### BRIEF DESCRIPTION OF THE DRAWINGS

FIG. 1 illustrates a perspective view of a system for enabling a user to exercise while providing input commands into a computing system, in accordance with embodiments of the present invention.

FIG. 2 illustrates a block diagram view of processing modules within the computing system of system in FIG. 1, in accordance with embodiments of the present invention.

FIG. 3 illustrates an algorithm describing a process used by the gesture interpreter module in FIG. 2, in accordance with embodiments of the present invention.

FIG. 4 illustrates an algorithm describing a process used by the input device data processing module in FIG. 2, in accordance with embodiments of the present invention.

FIG. 5 illustrates an algorithm describing a process used by the statistics module in FIG. 2, in accordance with embodiments of the present invention.

FIG. 6 illustrates an algorithm describing a method used by the control module in FIG. 2, in accordance with embodiments of the present invention.

FIG. 7 illustrates an algorithm describing a process for associating gestures and/or input devices with specified keyboard functions in the system of FIGS. 1 and 2, in accordance with embodiments of the present invention.

FIG. 8 illustrates a computer system comprising the computing system of FIGS. 1 and 2 used for implementing the associations between keyboard functions and input devices, in accordance with embodiments of the present invention.

### DETAILED DESCRIPTION OF THE INVENTION

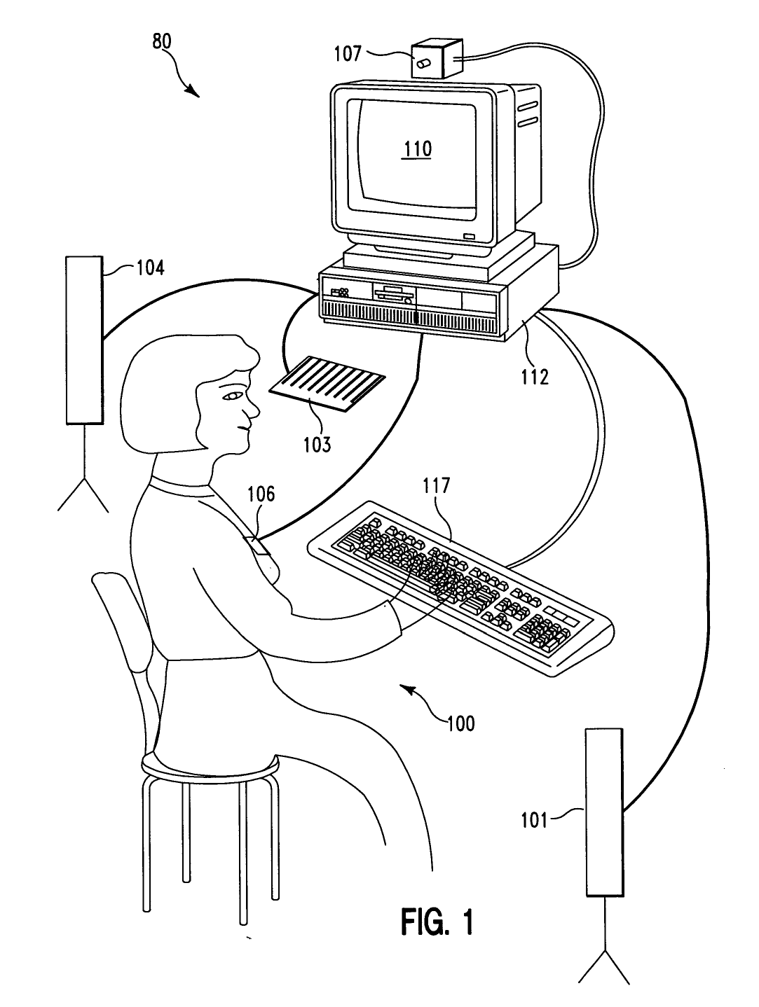

FIG. 1 illustrates a perspective view of a system 80 for enabling a user 100 to exercise while providing input commands into a computing system 112, in accordance with embodiments of the present invention. The system 90 allows a user 100 to activate various input devices 101, 103, 104, and 107 to provide input commands into the computing system 112 to perform various keyboard 117 commands during an exercise routine. The system 80 allows users (e.g., user 100) working on computers (e.g., computing system 112) to periodically engage in various physical movements to provide input into the computer (e.g., computing system 112). The various physical movements allow the user (e.g., user 100) to exercise while working at the computer. The various physical movements simulate various keyboard functions (e.g., enter function, tab function, control function, etc.). For example, the user 100 is typing on the computing system 112 using the keyboard 117. Most of keyboard 117 functions are executed using keys on the keyboard 117. Some of keyboard 117 functions are executed alternative input devices (e.g., input devices 101, 103, 104, 107) instead of using keys on the keyboard 117 (i.e., the use of the input devices 101, 103, 104, 107 are interpreted as keyboard functions by the computing system 112). A substitution example would be for the user 100 to use the keyboard 112 to execute all keyboard functions except the enter function. The user would use one of input devices 101, 103, 104, 107 to execute the enter function and therefore the user will exercise every time he/she executes the enter function. The system 80 additionally comprises a monitor 110 connected to the computing system. Each of input devices 101, 103, 104, and 107 and the keyboard 117 are connected to the computing system 112. Sensor system 106 comprises sensors for measuring vital signs and providing vital sign readings of the user 100. A vital sign reading may comprise any vital sign reading known to a person of ordinary skill in the art including, inter alia, a heart rate reading, a pulse rate reading, a respiratory rate reading, a body temperature reading, etc. The sensor system 106 is connected to the computer. Each of the input devices 101, 103, 104, and 107 are associated with a user (e.g., user 100) specified key on a keyboard 117. For example, input device 101 may be associated with the “enter” key on the keyboard 117. Therefore, the user 100 may use the keyboard 117 to input data into the computer system 112 and when the user 100 has to perform the “enter” function, the input device 101 may be activated (i.e., as part of an exercise routine) to provide an input command to activate the computing system 112 to perform the “enter” function instead of using the “enter” key. The “enter” key may also be used to perform the “enter” function thereby giving the user 100 multiple ways to perform the “enter” function.

Each of input devices 101, 103, 104 comprises a means for activating an input command. The means for activating an input command may comprise, inter alia, a switching device, a motion sensor, a touch sensor, etc. Each of the input devices 101, 103, 104 are activated during an exercise routine by physically contacting (i.e., by the user 100) the input devices 101, 103, 104. For example, the user 100 may hit his/her hand or foot on any of the input devices 101, 103, 104 to activate them and the computing system comprises an algorithm to accept the a signal from the activated input device and command the computing system 112 to perform an associated keyboard function. The associations between each of input devices 101, 103, 104 and specified keyboard functions may be provided by the user 100. The user 100 may perform various exercises using the input devices 101, 103, 104 while working at the computing system 112. Therefore the input devices 101, 103, 104 may be considered exercise devices. Each of the input devices 101, 103, 104 may be programmed to command the computing system 112 to perform an associated keyboard functions. For example, input device 101 may be programmed to command the computing system 112 to perform the “enter” function, input device 103 may be programmed to command the computing system 112 to perform the “escape” function, and input device 104 may be programmed to command the computing system 112 to perform the “shift” function. Therefore the user 100 performs various exercises every time he/she performs the “enter” function, the “escape” function, and the “shift” function. Each individual user (e.g., user 100) may program each of the input devices 101, 103, 104 to command the computing system 112 to perform different specified keyboard functions (i.e., associate input devices 101, 103, 104 with specified keyboard functions) in accordance with an exercise routine, thereby allowing each individual user to customize each of input devices 101, 103, 104 for specified keyboard functions and therefore a custom exercise routine. Additionally, each of the input devices 101, 103, 104 may be placed in various locations so that each individual user may activate the input devices 101, 103, 104 in custom locations further customizing an exercise routine.

Input device 107 is a video input device such as a video camera, etc. The input device 107 is adapted to send image data to the computing system 112. The user 100 programs the computing system 112 in accordance with an exercise routine to associate specified movements of the hands or body (for the user 100) with specified keys (i.e., specified keyboard functions) on the keyboard 117. For a first example, an exercise routine may call for the user 100 to execute a first specified movement with his/her arms (e.g., moving arms or legs backwards) so the user 100 may program the computing system 112 may to associate the first specified movement with the “enter” key on the keyboard 117. For a second example, an exercise routine may call for the user 100 to execute a second specified movement with his/her hands (e.g., hitting his/her hands on a desk) so the computing system 112 may be programmed to associate the second specified movement with the “escape” key on the keyboard 117. Therefore, in the preceding examples, the exercise routine comprises performing the first and second specified movements to activate the “enter” and “escape” commands. In order to program the computing system 112, the user executes various specified movements and the input device 107 records the various specified movements (e.g., first and second specified movements). The input device 107 transmits the recording of the various specified movements to the computing system 112. The user 100 may then decide how to associate each of the various specified movements to specified keys on the keyboard 117. The various specified movements associated with the specified keys on the keyboard 117 are stored in the computing system. When the user 100 uses the computing system 112, the input device 107 monitors the user movements. If the input device 107 detects specified movements stored in the computing device 112, those movements are associated with the specified keys on the keyboard 117 and the computing system executes the specified keyboard function associated with the specified key on the keyboard 117. Therefore, when a user 100 makes certain specified movements, specified keyboard functions are executed by the computing system 112. An exercise routine may be developed using the various physical movements required to activate input devices 101, 103, 104, and 107. As the user 100 uses the input devices 101, 103, 104, and 107 (i.e., exercises), the sensor system 106 measures vital signs of the user 100 and transmits vital sign readings to the computing system 112. The vital sign readings are analyzed by the computing system 112 (i.e., by a software application), and the software application determines how efficiently the user 100 is exercising and whether the user 100 should change his/her exercise routine in any way. If the software application determines that the user 100 is not exercising efficiently, it may change a force and/or a movement necessary to use/activate input devices 101, 103, 104, and 107 to execute input commands (keyboard functions) into the computing system 112. The software application may also automatically change the keys on the keyboard that are associated with each of the input devices 101, 103, 104, and 107 resulting in keys on the keyboard 117 that are used more often being associated with the exercise program.

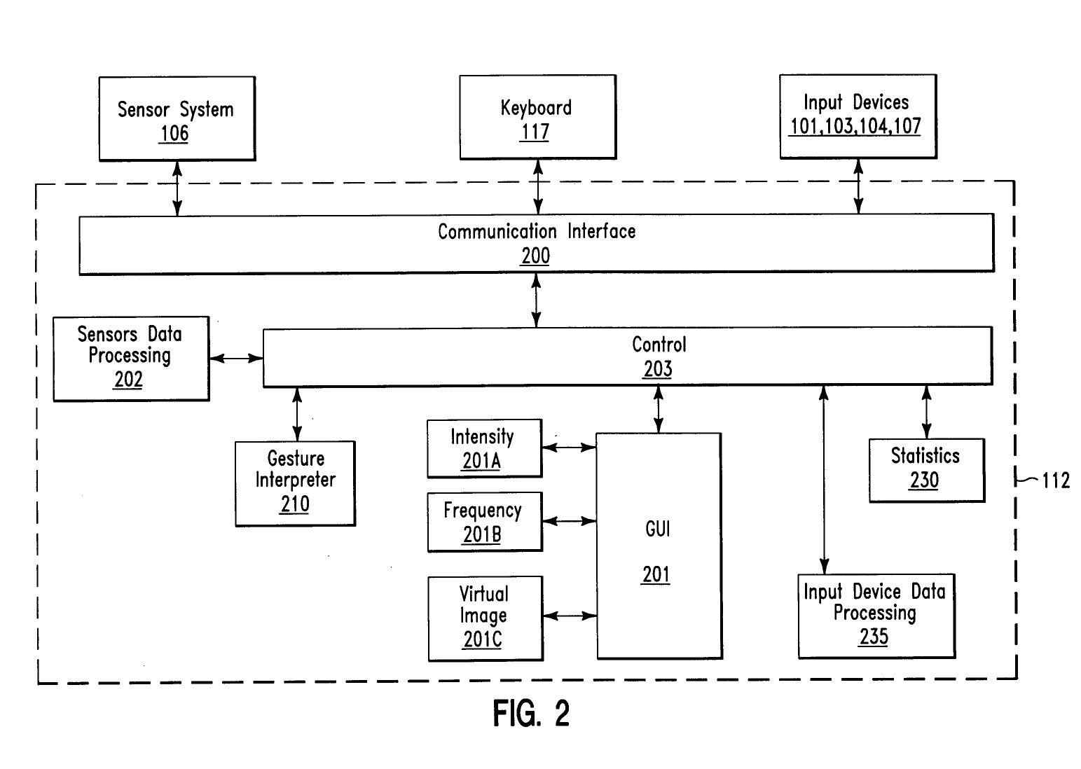

FIG. 2 illustrates a block diagram view of processing modules within the computing system 112 of system 80 in FIG. 1, in accordance with embodiments of the present invention. The system 80 in FIG. 2 is used to provide an exercise routine for users (e.g., user 100 in FIG. 1) of the computing system 112. Each of processing modules 200, 203, 201, 202, 201A, 201B, 201C, 210, 230, and 235 may comprise hardware modules, software, or any combination thereof. Communication interface module 200 interfaces the computer 112 to input devices 101, 103, 104, and 107, keyboard 117, and sensor system 106. The communication interface module 200 may comprise, inter alia, a wireless communication interface, an internet communication interface, an infrared communication interface, etc. Control module 203 controls all processing within the computing system 112. Input device data processing module 235 produces output data comprising user manipulations either via keyboard 117 or interpretation of data from input devices 101, 103, and 104. Gesture interpreter module 210 associates specified movements of the hands or body (for the user 100) with specified keys on the keyboard 117. The input device data processing module 235 sends the output data from the input device data processing module 235 to the statistics module 230. The gesture interpreter module 210 sends output data to the statistics module 230. The statistics module 230 processes the output data from the input device data processing module 235 and the gesture interpreter module 210 and derives user 100 statistics (e.g., about intensity of exercises, how the user feels etc.). Module 201 comprises a graphical user interface (GUI) that may be viewed on monitor 110 (in FIG. 1). The GUI allows the user 100 an ability to control the interface and associations between input devices 101, 103, 104, and 107 and specified keys on the keyboard 117. The user 100 may adjust an intensity of each of input devices 101, 103, 104, and 107 (i.e., to activate each of 101, 103, 104, and 107) required for the user to exercise in order to execute a corresponding keyboard command on the computer 112. The intensity may be adjusted using the intensity module 201A. The user 100 may adjust a frequency of exercises and repetition of exercises using the frequency module 201B. Virtual image module 201C is used to produce a virtual image of the keyboard 117 (i.e., on monitor 110 in FIG. 1) so that the user may associate the keyboard (i.e., keys on the keyboard 117) with each of input devices 101, 103, 104, and 107. The intensity module 201A, the frequency module 201B, and the virtual image module 201C allow the user to specify desired exercises and exercise goals. The desired exercise goals of the user may be inputted by the user 100 and stored in the computing system 112. The control module 200 may determine if the vital sign readings from the sensor system 106 match the desired exercise goals of the user 100. For example, a specified heart rate range may be associated with a cardio workout. Additionally, the control module 200 may automatically adjust (i.e., increase or decrease) an intensity of exercises in response to the vital sign readings. Sensors data processing module 202 processes the vital sign readings of the user from the sensor system 106. The sensors data processing module 202 sends the processed the vital sign readings to the control module 203. Additionally, the user 100 statistics (e.g., about intensity of exercises, how the user feels etc.) from the statistics module 230 are sent to the control module 203. The user 100 statistics (e.g. user activity and health history) from the statistics module 230 is updated continuously using the vital sign readings (e.g. heart rate when certain actions are performed). The control module 203 defines whether the user 100 activity that is recorded from sensor system 106 matches required user activity recorded in the statistics module 235.

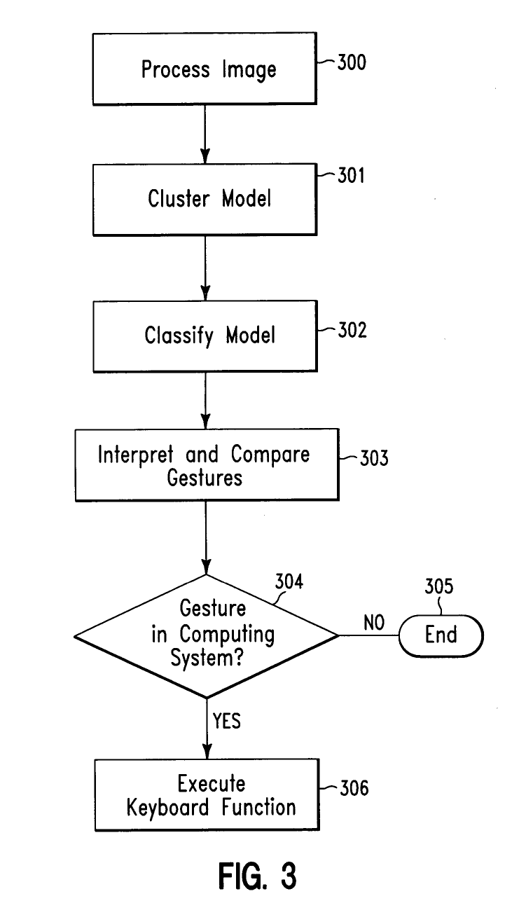

FIG. 3 illustrates an algorithm describing a process used by the gesture interpreter module 210 in FIG. 2, in accordance with embodiments of the present invention. The algorithm in FIG. 3 comprises using gestures/motions to execute keyboard functions that have been associated with specified gestures/motions and stored in the computing system 112. In step 300 video data (i.e., user 100 gestures associated with an exercise routine) is received from the input device 107 and formatted. In step 301, the received video data is compressed into clusters. In step, the gestures/motions that the user 100 makes are identified. For example, if the user 100 hits a table or motions backwards with both arms, these gestures are identified so that they may be translated into specified commands on the keyboard 117. In step 303, the gestures or motions are interpreted and compared to gestures stored in the computing system. In step 304 it is determined whether the gesture or motion made by the user 100 corresponds (i.e., is associated) to a gesture or motion stored in the computing system 112. If in step 304 it is determined that the gesture or motion made by the user 100 corresponds (i.e., is associated) to a gesture or motion stored in the computing system 112 then the associated keyboard function is executed by the computing system 112 in step 306. If in step 304 it is determined that the gesture or motion made by the user 100 does not correspond (i.e., is associated) to a gesture or motion stored in the computing system 112 then the process ends in step 305.

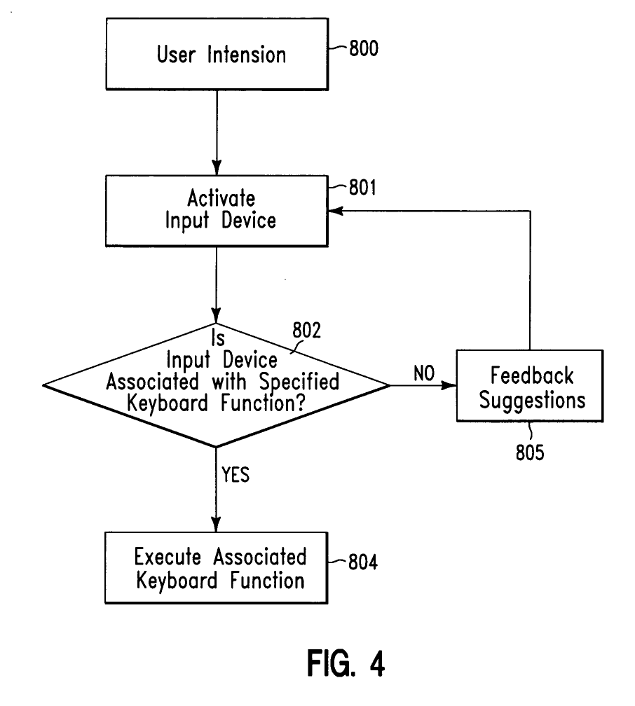

FIG. 4 illustrates an algorithm describing a process used by the input device data processing module 235 in FIG. 2, in accordance with embodiments of the present invention. The algorithm in FIG. 4 comprises physically contacting as part of an exercise routine, any of input devices 101, 103, or 104 to execute keyboard functions that have been associated any of input devices 101, 103, or 104 and stored in the computing system 112. In step 800, the user 100 decides to execute a specified keyboard function as part of an exercise routine, using one of the input devices 101, 103, or 104. In step 801, the user activates one of input devices 101, 103, or 104 which are thought to be associated with the specified keyboard function. In step 802, a determination is made as to whether the input device activated is associated with the specified keyboard function. If in step 802 it is determined that the input device activated is associated with the specified keyboard function then the specified keyboard function is executed by the computing system 112 in step 804. If in step 802 it is determined that the input device activated is not associated with the specified keyboard function then the computing system suggests the use of a different input device in step 805 and the process repeats step 801.

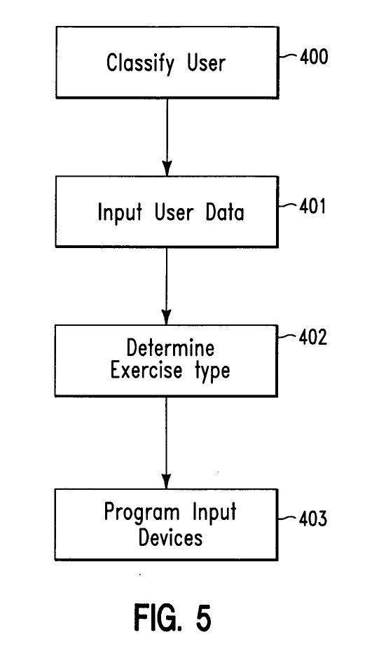

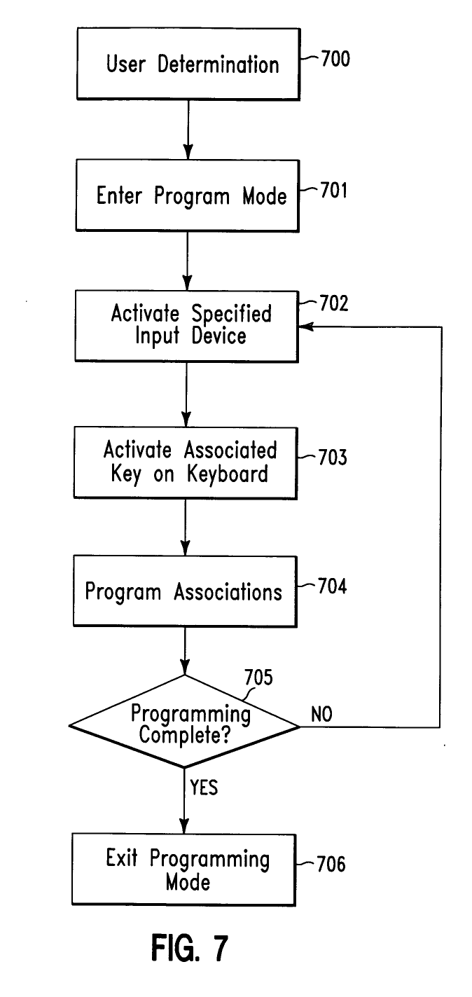

FIG. 5 illustrates an algorithm describing a process used by the statistics module 235 in FIG. 2, in accordance with embodiments of the present invention. The algorithm in FIG. 5 comprises developing an exercise routine for the user 100. In step 400 the user 100 is classified into a specified group. For example, the class may be based on, inter alia, age, gender, height, weight, health, etc. In step 401, input data regarding the user 100 is inputted into the computing system 112. For example, input data may comprise, inter alia, how many hours the user 100 plans to sit at the desk or work on a certain day, how often the user 100 takes breaks, etc. In step 402 the user 100 decides a type of exercise that he/she would like to do (e.g. hitting keyboard 117, hitting specified input devices 101, 103, 104, making specific gestures, etc.) associated with a specified type of exercise routine. For example, cardio exercise routine, fat burning exercise routine, muscle building exercise routine, etc. In step 403, the user programs the input devices in accordance with the specified exercise as described in the description of FIG. 7, supra.

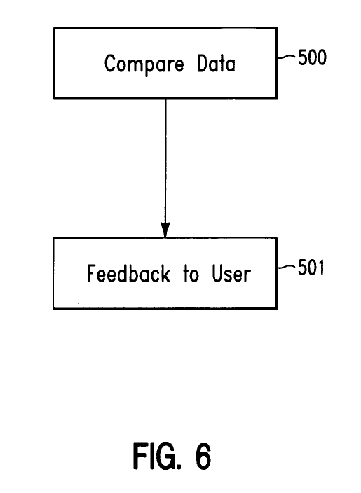

FIG. 6 illustrates an algorithm describing a method used by the control module 203 in FIG. 2, in accordance with embodiments of the present invention. In step 500, data received from the input devices 101, 103, 104, and 107 (e.g., type of exercise, intensity, etc.) is compared to user 100 input data (e.g., how often the user 100 takes breaks, how many hours the user 100 plans to sit at the desk or work on a certain day, etc). Any differences between the user 100 input data (e.g., user request) and data received from the input devices 101, 103, 104, and 107 produces user recommendations (i.e., feedback) as to how to change the exercise routine in step 501. The user recommendations are displayed for the user on the monitor 110 (see FIG. 1). User recommendations may comprise, inter alia, recommending new motions/gestures for associating with specified keys on the keyboard, increasing an intensity of the exercise, etc. Upon acceptance of the recommendation, the information is transmitted to the input devices 101, 103, 104, and 107 so that they react to a stronger or weaker force, respectively.

FIG. 7 illustrates an algorithm describing a process for associating gestures and/or input devices with specified keyboard functions in the system 80 of FIGS. 1 and 2, in accordance with embodiments of the present invention. In step 700, the user determines a specific gesture or input device to activate as part of an exercise routine and an association to a specific keyboard function on the keyboard 112. The specific gesture or input device to activate may be selected in response to a specified exercise. In step 701, the user 100 executes a programming mode in the computing system 112. Executing a programming mode may be accomplished by, inter alia, entering a user password, activating a switch, etc. In step 702, the user 100 executes the specific gesture for input device 107 or activates the specific input device (i.e., selected from input devices 101, 103, and 104). In step 703, the specific keyboard function on the keyboard 112 that will be associated with the specific gesture or input device 101, 103, and 104 is activated. In step 704, the computing system 112 programs associations between the specific keyboard function on the keyboard 112 and the specific gesture or specific input device (i.e., selected from input devices 101, 103, and 104). The associations are stored in the computing system 112. In step 705, the user determines if the programming is complete (i.e., any more associations to be programmed). If in step 705 the programming is complete then the user 100 exits the programming mode in step 706. If in step 705 the programming not complete (i.e., more associations to be programmed) then the process repeats step 702.

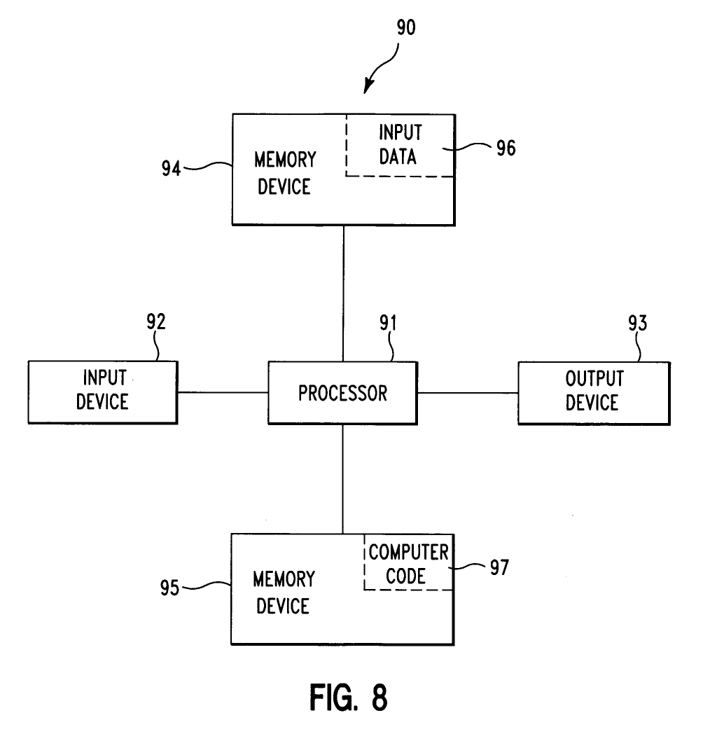

FIG. 8 illustrates a computer system 90 comprising the computing system 112 of FIGS. 1 and 2 used for implementing the associations between keyboard 117 functions and input devices 101, 103, 104, and 107, in accordance with embodiments of the present invention. The computer system 90 comprises a processor 91, an input device 92 coupled to the processor 91, an output device 93 coupled to the processor 91, and memory devices 94 and 95 each coupled to the processor 91. The input device 92 may be, inter alia, a keyboard, a mouse, etc. The output device 93 may be, inter alia, a printer, a plotter, a computer screen (e.g., monitor 110), a magnetic tape, a removable hard disk, a floppy disk, etc. The memory devices 94 and 95 may be, inter alia, a hard disk, a floppy disk, a magnetic tape, an optical storage such as a compact disc (CD) or a digital video disc (DVD), a dynamic random access memory (DRAM), a read-only memory (ROM), etc. The memory device 95 includes a computer code 97. The computer code 97 includes an algorithm for associating specific keyboard functions with specific input devices. The processor 91 executes the computer code 97. The memory device 94 includes input data 96. The input data 96 includes input required by the computer code 97. The output device 93 displays output from the computer code 97. Either or both memory devices 94 and 95 (or one or more additional memory devices not shown in FIG. 8) may comprise any of the algorithms of FIGS. 3-7 and may be used as a computer usable medium (or a computer readable medium or a program storage device) having a computer readable program code embodied therein and/or having other data stored therein, wherein the computer readable program code comprises the computer code 97. Generally, a computer program product (or, alternatively, an article of manufacture) of the computer system 90 may comprise said computer usable medium (or said program storage device).

While FIG. 8 shows the computer system 90 as a particular configuration of hardware and software, any configuration of hardware and software, as would be known to a person of ordinary skill in the art, may be utilized for the purposes stated supra in conjunction with the particular computer system 90 of FIG. 8. For example, the memory devices 94 and 95 may be portions of a single memory device rather than separate memory devices.

While embodiments of the present invention have been described herein for purposes of illustration, many modifications and changes will become apparent to those skilled in the art. Accordingly, the appended claims are intended to encompass all such modifications and changes as fall within the true spirit and scope of this invention.

What is claimed is:

1. A method, comprising:
providing a computing system connected to a video input apparatus, said video input apparatus adapted to send image data to said computing system;
developing an exercise routine for a user of said computing system;
associating a first function to be performed by said computing system with a first specified physical movement to be performed by said user of said computing system, said first specified physical movement being part of said exercise routine;
performing by said user, said first specified physical movement;
detecting by said video input apparatus, said first specified physical movement; and
transmitting by said video input apparatus to computing system, a first image of said first specified physical movement; and
performing by said computing system, said first function.

2. The method of claim 1, further comprising:
associating a second function to be performed by said computing system with a second specified physical movement to be performed by said user of said computing system, said second specified physical movement being part of said exercise routine;
performing by said user, said second specified physical movement;
detecting by said video input apparatus, said second specified physical movement; and
transmitting by said video input apparatus to computing system, a second image of said second specified physical movement; and
performing by said computing system, said second function.

3. The method of claim 2, wherein said first specified physical movement and said second specified physical movement each comprise a different physical movement, and wherein said first function and said second function each comprise a different function.

4. The method of claim 1, further comprising:
providing a plurality of sensors connected to said computing system;
receiving by each of said sensors, a different vital sign reading of said user;
modifying said exercise routine in response to said receiving;
associating said first function to be performed by said computing system with a third specified physical movement to be performed by said user of said computing system in response to said modified exercise routine;
performing by said user, said third specified physical movement as part of said modified exercise routine;
detecting by said video input apparatus, said third specified physical movement; and
transmitting by said video input apparatus to computing system, a third image of said third specified physical movement; and
performing by said computing system, said first function.

5. The method of claim 4, wherein each said different vital sign reading of said user is selected from the group consisting of a pulse rate reading, a respiratory rate reading, a body temperature reading, and a blood pressure reading.

6. The method of claim 1, further comprising providing an input device connected to said computing system, said input device adapted to trigger said computing system to perform said first function.

7. The method of claim 6, wherein said input device is selected from the group consisting of a keyboard, a keypad, and a computer mouse.

8. A method, comprising:
providing a computing system connected to a first exercise apparatus, said first exercise apparatus adapted to send a first signal to said computing system upon using said first exercise apparatus;
developing an exercise routine for a user of said computing system;
associating a first function to be performed by said computing system with said first signal from said first exercise apparatus, said first exercise apparatus being associated with said exercise routine;
using by a user, said first exercise apparatus as part of said exercise routine;
receiving by said computing system, said first signal; and
performing by said computing system, said first function.

9. The method of claim 8, further comprising:
providing a second exercise apparatus connected to said computing system, wherein said second exercise input apparatus is adapted to send a second signal to said computing system upon using said second exercise apparatus, said second exercise apparatus being associated with said exercise routine;
associating a second function to be performed by said computing system with said second signal from said second exercise apparatus;
using by said user, said second exercise apparatus as part of said exercise routine;
receiving by said computing system, said second signal; and
performing by said computing system, said second function.

10. The method of claim 9, wherein said first function and said second function each comprise a different function, and wherein said first exercise apparatus said second exercise apparatus are each for performing a different exercise.

11. The method of claim 8, further comprising:
providing a third exercise apparatus connected to said computing system and a plurality of sensors connected to said computing system, wherein said third exercise apparatus is adapted to send a third signal to said computing system upon using said third exercise apparatus;
receiving by each of said sensors, a different vital sign reading of a user; and
modifying said exercise routine in response to said receiving;
associating said first function to be performed by said computing system with said third signal from said third exercise apparatus in response to said modified exercise routine;
using by said user, said third exercise apparatus as part of said modified exercise routine;
receiving by said computing system, said third signal; and
performing by said computing system, said first function.

12. The method of claim 11, wherein each said different vital sign reading of said user is selected from the group consisting of a pulse rate reading, a respiratory rate reading, a body temperature reading, and a blood pressure reading.

13. The method of claim 8, further comprising providing an input device connected to said computing system, said input device adapted to trigger said computing system to perform said first function.

14. The method of claim 13, wherein said input device is selected from the group consisting of a keyboard, a keypad, and a computer mouse.

15. A computing system comprising a processor coupled to a computer-readable memory unit, said memory unit containing instructions that when executed by the processor implement a method for performing by said computing system, at least one function, said method comprising;
providing a video input apparatus connected to said computing system, said video input apparatus adapted to send image data to said computing system;
developing an exercise routine for a user of said computing system;
associating a first function to be performed by said computing system with a first specified physical movement to be performed by said user of said computing system, said first specified physical movement being part of said exercise routine;
performing by said user, said first specified physical movement;
detecting by said video input apparatus, said first specified physical movement; and
transmitting by said video input apparatus to said computing system, a first image of said first specified physical movement; and
performing by said computing system, said first function.

16. The computing system of claim 15, wherein said method further comprises:
associating a second function to be performed by said computing system with a second specified physical movement to be performed by said user of said computing system, said second specified physical movement being part of said exercise routine;
performing by said user, said first specified physical movement;
detecting by said video input apparatus, said second specified physical movement; and
transmitting by said video input apparatus to computing system, a second image of said second specified physical movement; and
performing by said computing system, said second function.

17. The computing system of claim 16, wherein said first specified physical movement and said second specified physical movement each comprise a different physical movement, and wherein said first function and said second function each comprise a different function.

18. The computing system of claim 15, wherein said method further comprises:
providing a plurality of sensors connected to said computing system;
receiving by each of said sensors, a different vital sign reading of said user;
modifying said exercise routine in response to said receiving;
associating said first function to be performed by said computing system with a third specified physical movement to be performed by said user of said computing system in response to said modified exercise routine;
performing by said user, said third specified physical movement as part of said modified exercise routine;
detecting by said video input apparatus, said third specified physical movement; and
transmitting by said video input apparatus to computing system, a third image of said third specified physical movement; and
performing by said computing system, said first function.

19. The computing system of claim 15, wherein each said different vital sign reading of said user is selected from the group consisting of a pulse rate reading, a respiratory rate reading, a body temperature reading, and a blood pressure reading.

20. The computing system of claim 15, wherein said method further comprises:
providing an input device connected to said computing system, said input device adapted to trigger said computing system to perform said first function.

21. The computing system of claim 15, wherein said input device is selected from the group consisting of a keyboard, a keypad, and a computer mouse.

22. A computing system comprising a processor coupled to a computer-readable memory unit, said memory unit containing instructions that when executed by the processor implement a method for performing by said computing system, at least one function, said method comprising;
providing a first exercise apparatus connected to said computing system, said first exercise apparatus adapted to send a first signal to said computing system upon using said first exercise apparatus;
developing an exercise routine for a user of said computing system;
associating a first function to be performed by said computing system with said first signal from said first exercise apparatus, said first exercise apparatus being associated with said exercise routine;
using by a user, said first exercise apparatus as part of said exercise routine;
receiving by said computing system, said first signal; and
performing by said computing system, said first function.

23. The computing system of claim 22, further comprising:
providing a second exercise apparatus connected to said computing system, wherein said second exercise input apparatus is adapted to send a second signal to said computing system upon using said second exercise apparatus, said second exercise apparatus being associated with said exercise routine;
associating a second function to be performed by said computing system with said second signal from said second exercise apparatus;
using by said user, said second exercise apparatus as part of said exercise routine;
receiving by said computing system, said second signal; and
performing by said computing system, said second function.

24. The computing system of claim 23, wherein said first function and said second function each comprise a different function, and wherein said first exercise apparatus said second exercise apparatus are each for performing a different exercise.

25. The computing system of claim 22, wherein said method further comprises:
providing a third exercise apparatus connected to said computing system and a plurality of sensors connected to said computing system, wherein said third exercise input apparatus is adapted to send a third signal to said computing system upon using said second exercise;
receiving by each of said sensors, a different vital sign reading of a user;
modifying said exercise routine in response to said receiving;
associating said first function to be performed by said computing system with said third signal from said third exercise apparatus in response to said modified exercise routine;
using by said user, said third exercise apparatus as part of said modified exercise routine;
receiving by said computing system, said third signal; and
performing by said computing system, said first function.

26. The computing system of claim 25, wherein each said different vital sign reading of said user is selected from the group consisting of a pulse rate reading, a respiratory rate reading, a body temperature reading, and a blood pressure reading.

27. The computing system of claim 22, wherein said method further comprises:
providing an input device connected to said computing system, said input device adapted to trigger said computing system to perform said first function.

28. The computing system of claim 27, wherein said input device is selected from the group consisting of a keyboard, a keypad, and a computer mouse.
